(19) United States
(12) Patent Application Publication
(10) Pub. No.: US 2017/0136299 A1
(43) Pub. Date: May 18, 2017

(54) MUSCLE ACTIVITY AMOUNT DETERMINING DEVICE, MUSCLE ACTIVITY AMOUNT DETERMINING METHOD, RECORDING MEDIUM, AND METHOD

(71) Applicant: Panasonic Intellectual Property Management Co., Ltd., Osaka (JP)

(72) Inventors: SOUKSAKHONE BOUNYONG, Nara (JP); SHINOBU ADACHI, Nara (JP); JUN OZAWA, Nara (JP)

(21) Appl. No.: 15/297,408

(22) Filed: Oct. 19, 2016

(30) Foreign Application Priority Data
Nov. 12, 2015 (JP) 2015-221845

(51) Int. Cl. A63B 24/00; A61B 5/00; A63B 23/04; A61B 5/11

(52) U.S. Cl. CPC A63B 24/0062; A61B 5/1128; A61B 5/1127; A61B 5/1118; A61B 5/4519; A63B 23/0476; A63B 2230/60; A63B 2220/836; A63B 2220/807

(57) ABSTRACT

A muscle activity amount determining device includes a shape obtainer that obtains a shape of a muscle of a user when the user is doing certain exercise; a position identifying unit that identifies a position of the muscle of the user when the user is doing the exercise; a determination circuit that refers to a determination reference that indicates a corresponding relationship between the position and shape of the muscle and an activity amount of the muscle, and determines the activity amount of the muscle using the position of the muscle identified by the position identifying unit and the shape of the muscle obtained by the shape obtainer; and an outputter that outputs the activity amount, determined by the determination circuit.

### BACKGROUND

1. Technical Field

The present disclosure relates to techniques for determining a muscle activity amount during exercise.

2. Description of the Related Art

Measurement of a muscle activity is important because it serves as an index for improving and evaluating the performance of exercise. One representative method of measuring a muscle activity uses surface electromyography (EMG) (EMG signals measured on a body surface near the muscle of interest). For example, Japanese Unexamined Patent Application Publication No. 2000-316827 discloses the following method of measuring a muscle activity using surface EMG. That is, an electrode is attached to the surface of the muscle of interest of a user, and the user does exercise with the maximum intensity. At that time, the maximum activity muscle potential detected by the electrode serves as reference measurement data. The ratio of the magnitude of EMG signals successively measured during the user's exercise to the magnitude of the reference measurement data is evaluated as the user's muscle activity.

However, the method of measuring the muscle activity using surface EMG is sometimes unable to correctly measure the muscle activity due to noise generated by the electrode being displaced or coming off because of the user's exercise. For example, there is clothing in which a conductive fabric is embedded as an electrode. When a user wears this clothing, EMG signals are detectable. When such clothing is used, though the user can easily wear the clothing, the electrode is often displaced or comes off since the electrode is not fixed to the skin. In short, the method using surface EMG requires the electrode to be mounted on the user such that a state where the electrode and the skin are reliably connected to each other is always maintained in order to perform accurate measurement. However, it is not always easy to mount the electrode on the user in such a manner. This problem is particularly striking in heavy exercise.

### SUMMARY

One non-limiting and exemplary embodiment provides a muscle activity amount determining device that uses an easy-to-wear measuring instrument and that can highly accurately determine the activity amount of a muscle even during heavy exercise.

In one general aspect, the techniques disclosed here feature a muscle activity amount determining device including: a shape obtainer that obtains a shape of a muscle of a user when the user is doing certain exercise; a position identifying unit that identifies a position of the muscle of the user when the user is doing the exercise; a determination circuit that refers to a determination reference that indicates a corresponding relationship between the position and shape of the muscle and an activity amount of the muscle, and determines the activity amount of the muscle using the position of the muscle identified by the position identifying unit and the shape of the muscle obtained by the shape obtainer; and an outputter that outputs the activity amount of the muscle, the activity amount being determined by the determination circuit.

According to the present disclosure, the measuring instrument is easy to wear, and the muscle activity amount can be highly accurately determined even during heavy exercise.

It should be noted that general or specific embodiments may be implemented as a system, a method, an integrated circuit, a computer program, a computer-readable recording medium, or any selective combination thereof. The computer-readable recording medium encompasses a non-volatile recording medium such as a CD-ROM (Compact Disc-Read Only Memory).

Additional benefits and advantages of the disclosed embodiments will become apparent from the specification and drawings. The benefits and/or advantages may be individually obtained by the various embodiments and features of the specification and drawings, which need not all be provided in order to obtain one or more of such benefits and/or advantages.

### BRIEF DESCRIPTION OF THE DRAWINGS

FIG. 1A is a schematic diagram illustrating the environment of an experiment forming the basis of the present disclosure;

FIG. 1B is a sectional view illustrating an exemplary cross-section of the left thigh of a test subject;

FIG. 1C is a diagram illustrating exemplary marker angles;

FIG. 2 is a conceptual diagram representing pedal positions;

FIG. 3A is a graph illustrating an example of the relaxant average and the contractile average of each marker angle;

FIG. 3B is a graph illustrating an example of the relaxant average and the contractile average of each marker angle;

FIG. 3C is a graph illustrating an example of the relaxant average and the contractile average of each marker angle;

FIG. 3D is a graph illustrating an example of the relaxant average and the contractile average of each marker angle;

FIG. 4A is a graph illustrating an example of the relaxant average and the contractile average of each marker angle;

FIG. 4B is a graph illustrating an example of the relaxant average and the contractile average of each marker angle;

FIG. 5 is a graph illustrating an example of the relaxant average and the contractile average of each marker angle;

FIG. 6 is a block diagram illustrating an exemplary functional configuration of a muscle activity amount determining device according to a first embodiment;

FIG. 7 is a schematic diagram illustrating an exemplary appearance of the muscle activity amount determining device according to the first embodiment;

FIG. 8 is a diagram illustrating an exemplary conversion table for converting a tilt angle of a thigh to a pedal position according to the first embodiment;

FIG. 9 is a diagram illustrating exemplary determination references according to the first embodiment;

FIG. 10 is a flowchart illustrating an exemplary operation of the muscle activity amount determining device according to the first embodiment;

FIG. 11 is a diagram illustrating an exemplary result of the operation of the muscle activity amount determining device according to the first embodiment;

FIG. 12 is a diagram illustrating an exemplary output of a muscle activity amount according to the first embodiment;

FIG. 13 is a diagram illustrating the positions of electrodes included in a pair of shorts;

FIG. 14 is a graph illustrating exemplary EMG signals measured in the experiment;

FIG. 15 is a diagram illustrating an exemplary combination of an EMG sensor and a muscle shape measuring device;

FIG. 16 is a block diagram illustrating an exemplary functional configuration of a muscle activity amount determining device according to a second embodiment; and

FIG. 17 is a flowchart illustrating an exemplary operation of the muscle activity amount determining device according to the second embodiment.

### DETAILED DESCRIPTION

### Underlying Knowledge Forming Basis of the Present Disclosure

The inventors of the present disclosure point out that, with regard to the muscle activity measurement mentioned in “Description of the Related Art”, it is not easy to mount the electrode in the method using surface EMG. In view of this problem, the inventors pay attention to the fact that the shape of a muscle changes when the muscle is caused to contract. The inventors examine determination of a muscle activity amount from the muscle shape measurable using a measuring instrument that is easier to mount than using surface EMG.

In this examination, the inventors of the present disclosure have conducted an experiment for finding a reference for determining a muscle activity amount on the basis of the muscle shape. The experiment will be described in detail with reference to the drawings.

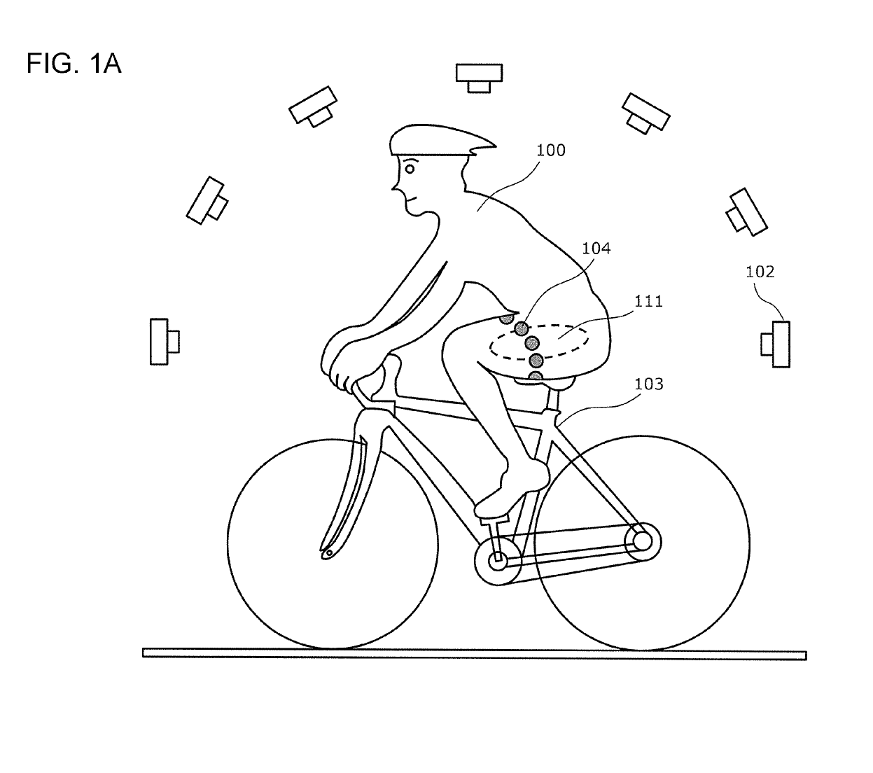

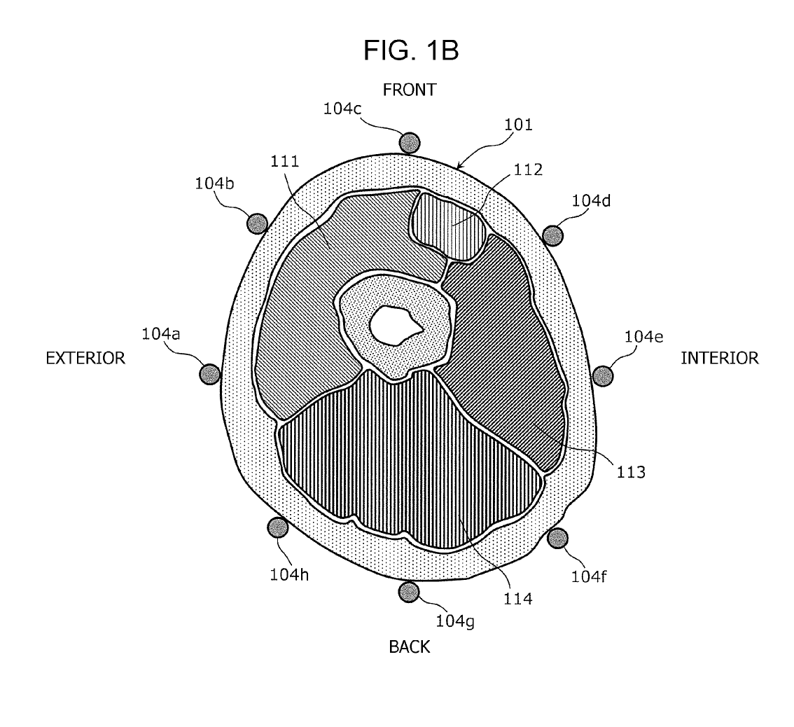

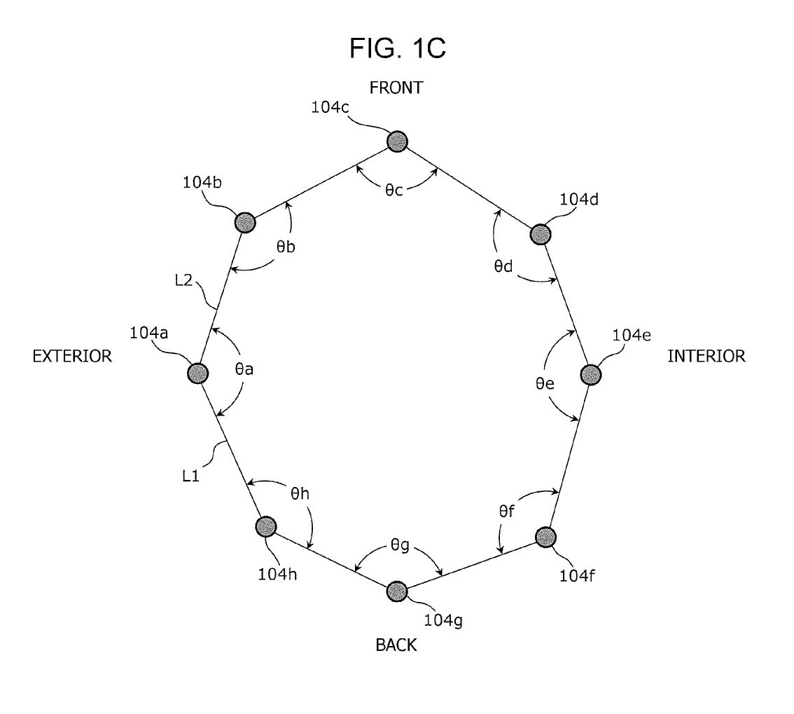

FIG. 1A is a schematic diagram describing the environment of the experiment. In this experiment, cameras 102 are used to take pictures of a test subject 100 riding on a bicycle 103 in the environment illustrated in FIG. 1A. The test subject is a male adult.

Eight markers 104 are attached around the left thigh of the test subject 100.

The cameras 102 are seven infrared cameras (VENUS3D, Nobby Tech. Ltd.). The sampling frequency is 240 Hz. The cameras 102 capture the movement of the eight markers 104.

The frame of the bicycle 103 is fixed using a cycle trainer such that the bicycle 103 stands erect. The test subject 100 wears dedicated shoes that can be locked with the pedals.

FIG. 1B is a sectional view illustrating an exemplary cross-section 101 of the left thigh of the test subject 100. As illustrated in FIG. 1B, markers 104a to 104h, which serve as the above-mentioned eight markers 104, are attached at substantially equal distances around the left thigh of the test subject 100. The cross-section 101 includes a vastus lateralis muscle 111, a rectus femoris muscle 112, a vastus medialis muscle 113, and a hamstring 114.

The shape of the vastus lateralis muscle 111 and the rectus femoris muscle 112 in the cross-section 101 is reflected in the positions of the markers 104a, 104b, and 104c. The shape of the vastus medialis muscle 113 in the cross-section 101 is reflected in the positions of the markers 104d and 104e. The shape of the hamstring 114 in the cross-section 101 is reflected in the positions of the markers 104f, 104g, and 104h. In this experiment, for practical convenience, the shape of a muscle is represented by an angle formed by a polygonal line connecting three adjacent markers. Hereinafter, the angle may simply be referred to as the angle of a marker or a marker angle. Note that a marker angle serves merely as an example for representing the shape of a muscle, and representing the shape of a muscle is not limited to this example.

FIG. 1C is a diagram illustrating exemplary marker angles. For example, there are the marker 104a, and the markers 104h and 104b adjacent to the marker 104a. A first straight line L1 connecting the marker 104a and the marker 104h and a second straight line L2 connecting the marker 104a and the marker 104b forms an angle, which is an angle θa of the marker 104a. Likewise, the angles of the markers 104b to 104h are defined.

The test subject 100 in the experiment has the following task. The pedals of the bicycle 103 are fixed to the positions at 6, 3, 12, and 9 o'clock of an analog clock. The test subject 100 places his/her feet on the pedals, fixed to the respective positions, and takes a posture of pedaling the bicycle 103. The test subject 100 does two sets of an operation of contracting the muscles of the thighs for five seconds with the maximal muscle strength and relaxing the muscles for five seconds without applying any strength.

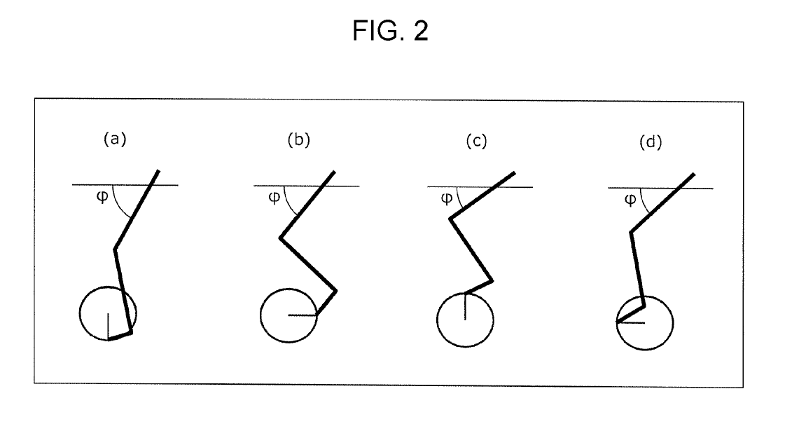

FIG. 2 is a conceptual diagram representing the pedal positions. This diagram corresponds to a lateral view of the left leg of the test subject 100 seen from the outside. Parts (a) to (d) of FIG. 2 represent the pedal positions at 6, 3, 12, and 9 o'clock, respectively. At each pedal position, the left thigh of the test subject 100 is tilted by an angle φ from the horizontal plane. Each pedal position corresponds to the position of a muscle of the test subject 100, who is a user, when the test subject 100 is doing certain exercise (turning the pedals of the bicycle 103 in this experiment, and this may simply be referred to as pedaling).

At all the pedal positions, that is, a plurality of positions of muscles while the test subject 100 is pedaling, the movement of the markers 104a to 104h is captured from the test subject 100 who is executing the above-mentioned task, and temporal changes of the angles θa to θh are obtained. For each of the angles θa to θh, an average (contractile average) for a total of ten seconds in a muscle contraction state where the test subject 100 is exerting the maximal muscle strength and an average (relaxant average) for a total of ten seconds in a muscle relaxation state without applying any strength are calculated.

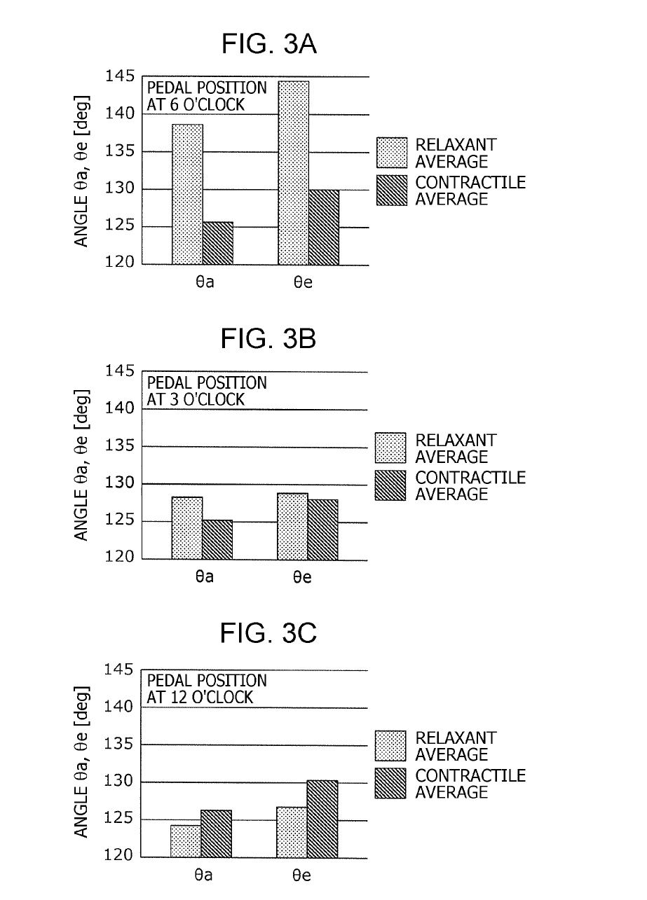

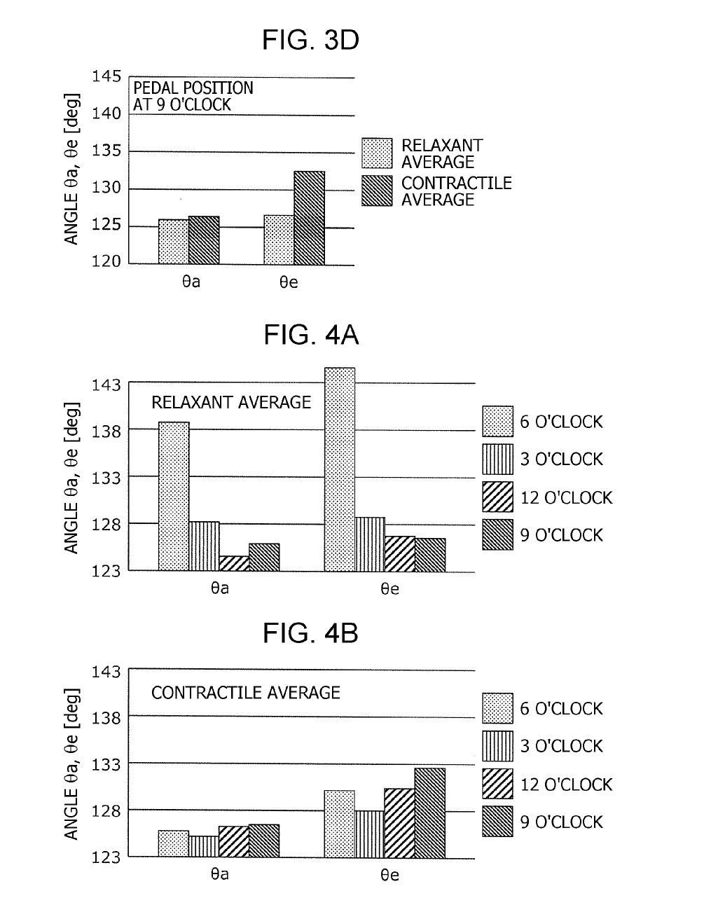

FIGS. 3A to 3D are graphs illustrating an example of the relaxant average and the contractile average calculated for the angle θa and the angle θe in the task executed at the pedal positions at 6, 3, 12, and 9 o'clock, respectively. In FIGS. 3A to 3D, the abscissa axis differentiates the angles θa and θe, and the ordinate axis represents θa and θe.

As seen in FIGS. 3A to 3D, whether the angles θa and θe increase or decrease in accordance with muscle contraction is different depending on the pedal position. For example, when the pedal position is at 6 and 3 o'clock (FIGS. 3A and 3B), the contractile average is less than the relaxant average for both the angle θa and the angle θe. In short, the angles θa and θe decrease in accordance with muscle contraction. When the pedal position is at 12 and 9 o'clock (FIGS. 3C and 3D), the contractile average is greater than the relaxant average for both the angle θa and the angle θe. In short, the angles θa and θe increase in accordance with muscle contraction.

FIGS. 4A and 4B are graphs comparing the relaxant average and the contractile average of each of the angle θa and the angle θe illustrated in FIGS. 3A to 3D according to each pedal position. In FIGS. 4A and 4B, the abscissa axis differentiates the angles θa and θe, and the ordinate axis represents θa and θe.

As seen in FIGS. 4A and 4B, the relaxant average or the contractile average of the same angle θa or the same angle θe has a magnitude that is different depending on the pedal position. For example, in FIG. 4A, although the test subject 100 is not applying strength to the thigh, the magnitude of the relaxant average of both the angle θ and the angle θe changes depending on the pedal position. Furthermore, the comparison between FIGS. 4A and 4B clarifies that, for both the angle θa and the angle θe, a variation pattern of the relaxant average is different from a variation pattern of the contractile average in accordance with the pedal position.

Such a tendency holds true for the angle θa and the angle θe, but a different tendency is observed for the angle θb.

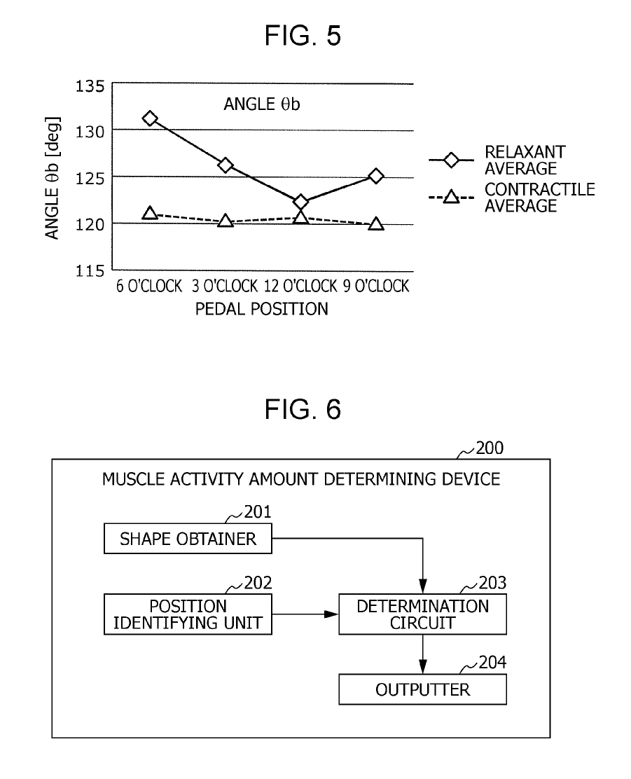

FIG. 5 is a graph illustrating an example of the relaxant average and the contractile average of the angle θb calculated in the above-described experiment. In FIG. 5, the abscissa axis represents the pedal position, and the ordinate axis represents the angle θb.

As seen in FIG. 5, unlike the angle θa or θe, the angle θb decreases in accordance with muscle contraction, regardless of the pedal position.

The inventors of the present disclosure have newly found out that the muscle shape changes not only in muscle contraction, but also in accordance with the muscle position during certain exercise done by the test subject 100 (for example, the pedal position in pedaling). Thus, the inventors of the present disclosure propose to determine the activity amount of a muscle of the test subject 100 (including displaying the degree or tendency merely indicating that the activity amount is great or small) in accordance with a determination reference indicating the corresponding relationship between the position and shape of the muscle and the activity amount of the muscle.

For example, the following determination reference is obtained from the results illustrated in FIGS. 3A to 3D. That is, when the pedal position is within a range including 6 o'clock or 3 o'clock, if the angle θa or the angle θe is less than a threshold defined between the relaxant average and the contractile average, it is determined that the activity amount of the muscle reflected in the marker 104a or the marker 104e is great. When the pedal position is near 12 o'clock or 9 o'clock, if the angle θa or the angle θe is greater than or equal to a threshold defined between the relaxant average and the contractile average, it is determined that the activity amount of the muscle reflected in the marker 104a or the marker 104e is great.

Alternatively, for example, the following determination reference is obtained from the results illustrated in FIG. 5. That is, when the pedal position is within a range including any of 6, 3, 12, and 9 o'clock, if the angle θb is less than a threshold defined between the relaxant average and the contractile average, it is determined that the activity amount of the muscle reflected in the marker 104b is great.

The cause of the influence of the position of a muscle during exercise on the shape of the muscle is that a gravity component or an external force applied to the muscle changes in accordance with the tilt angle (φ in FIG. 2) of the muscle with respect to the horizontal plane or the angle of a joint connected to the muscle, and this is not a phenomenon unique to pedaling. Therefore, the concept of determining the activity amount of a muscle in accordance with a determination reference represented by the position and shape of the muscle is not limited to pedaling and is applicable to various types of exercise, such as walking and weight training.

Hereinafter, a muscle activity amount determining device and a muscle activity amount determining method according to an aspect of the present disclosure will be specifically described with reference to the drawings.

All of embodiments described below indicate specific examples of the present disclosure. Numerical values, shapes, materials, elements, the arrangement positions and connection configuration of elements, steps, the order of steps, and so forth indicated in the following embodiments are only exemplary and are not construed to limit the present disclosure. Furthermore, among the elements in the following embodiments, elements that are not defined in an independent claim indicating the broadest concept are described as arbitrary elements.

### First Embodiment

FIG. 6 is a block diagram illustrating an exemplary functional configuration of a muscle activity amount determining device according to a first embodiment. A muscle activity amount determining device 200 illustrated in FIG. 6 is a device that determines the activity amount of a muscle during certain exercise by using the position and shape of the muscle. The muscle activity amount determining device 200 includes a shape obtainer 201, a position identifying unit 202, a determination circuit 203, and an outputter 204.

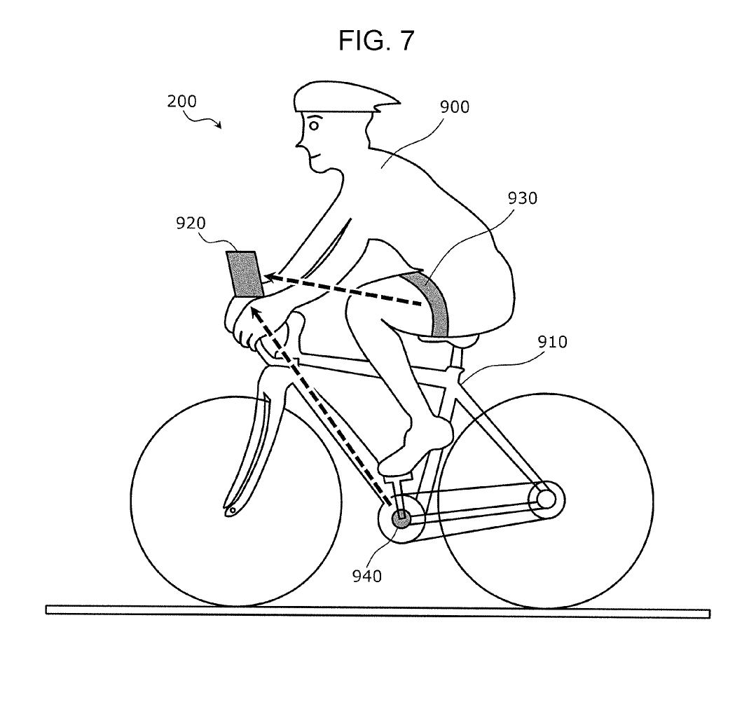

FIG. 7 is a schematic diagram illustrating an exemplary appearance of the muscle activity amount determining device 200. FIG. 7 illustrates an example of the muscle activity amount determining device 200, which determines the muscle activity amount of a thigh of a user 900 pedaling a bicycle 910. In this example, the muscle activity amount determining device 200 includes an information terminal 920 (such as a cycle computer or a smart phone), a sensor unit 930 worn around the thigh of the user 900, and a sensor unit 940 attached to the bicycle 910.

The shape obtainer 201 obtains the shape of a muscle of the user 900, and notifies the determination circuit 203 of information indicating the obtained shape. The shape obtainer 201 obtains the shape of a muscle of the user 900 every certain cycle.

An example of the shape of a muscle of the user 900 is the shape of the muscle in a cross-section including muscles of the limbs and the body of the user 900.

The shape of the muscle may be represented by an angle formed by a polygonal line connecting three points (three adjacent markers in the above-described experiment) defined on the outer circumference of the cross-section of the limbs and the body (such as the body, an arm, or a leg).

As the hardware of the shape obtainer 201, for example, motion capture or a strain sensor to be worn by the user 900 may be used.

In the case of using motion capture, like the above-mentioned experiment, on the basis of third-dimensional coordinates of a first marker, a second marker, and a third marker placed on a leg of the user 900 (specifically, a first marker placed on a muscle, and a second marker and a third marker placed adjacent to the first marker), an angle formed by a first straight line connecting the first marker and the second marker, and a second straight line connecting the first marker and the third marker is obtained as a marker angle (that is, the shape of the muscle). In this case, the markers serve as examples of a sensor included in the shape obtainer 201. Note that a plane on which the first maker, the second marker, and the third marker are placed may be a face orthogonal to the long-axis direction of the leg.

In the case of using a strain sensor, the curvature of a body surface, which reflects the shape of a muscle, is detected in accordance with the magnitude of a strain received by the strain sensor. The curvature of the body surface corresponds to the marker angle and represents the shape of the muscle. The sensor unit 930 in FIG. 7 is an example of the shape obtainer 201 using a strain sensor.

The shape obtainer 201 transmits information representing the shape of the muscle, which is obtained using motion capture or a strain sensor, to the determination circuit 203 wirelessly, for example.

The position identifying unit 202 periodically identifies the position of a muscle during certain exercise done by the user 900, and notifies the determination circuit 203 of information representing the identified position.

An example of the certain exercise done by the user 900 is pedaling a bicycle, which is turning the pedals of the bicycle. This exercise is called pedaling of a bicycle. A bicycle includes a cycle trainer. Examples of the position of a muscle of the user 900 are pedal positions (positions at 6, 3, 12, and 9 o'clock in the above-mentioned experiment).

As the position identifying unit 202, for example, motion capture, a rotation angle sensor that detects the rotation angle of a chain wheel of the bicycle 910, or a tilt sensor that detects the tilt angle φ (see FIG. 2) of a thigh of the user 900 from the horizontal plane may be used. The tilt sensor may be a geomagnetic sensor or an acceleration sensor fixed to a thigh of the user 900, or a combination thereof.

In the case of using a rotation angle sensor, a rotation angle obtained by the rotation angle sensor serves as a pedal position (in other words, an angle in one rotation of pedaling). The sensor unit 940 in FIG. 7 is an example of the position identifying unit 202 using a rotation angle sensor.

In the case of using a tilt sensor, the position identifying unit 202 calculates a pedal position from the tilt angle φ of the thigh, detected by the tilt sensor. The position identifying unit 202 converts the tilt angle φ to a pedal position by referring to, for example, a conversion table prepared in advance.

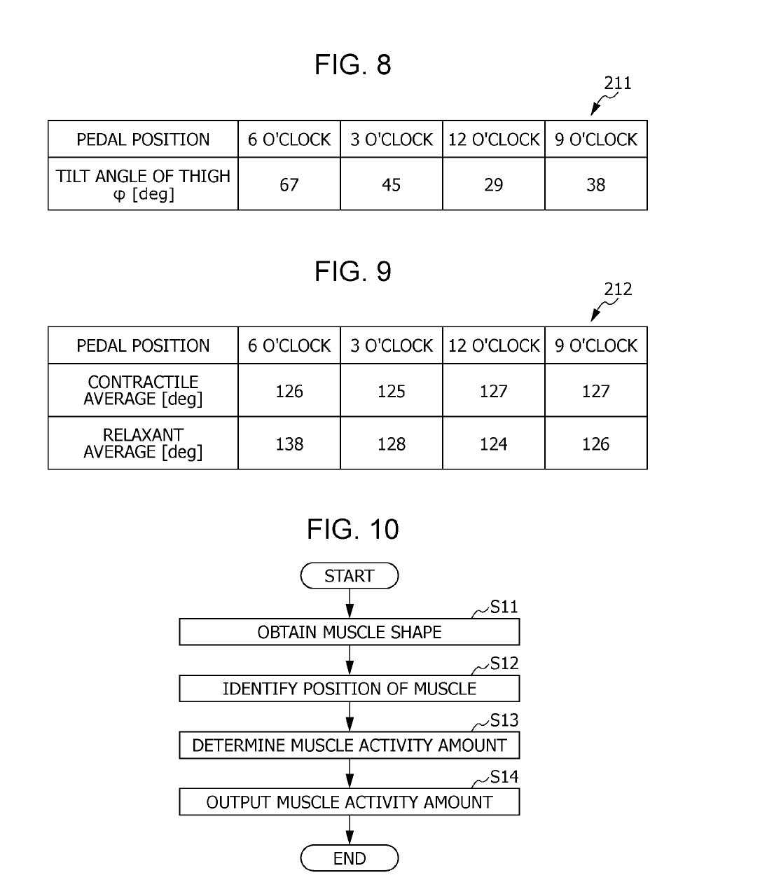

FIG. 8 is a diagram illustrating an example of a conversion table 211 for converting the tilt angle φ to a pedal position. In the conversion table 211, the results of measuring the tilt angle φ of the thigh of the user 900 from the horizontal plane at the pedal positions at 6, 3, 12, and 9 o'clock are recorded in advance.

When an angle substantially identical to (for example, within the range of ±10% of) each tilt angle φ indicated in the conversion table 211 is detected by the tilt sensor, the position identifying unit 202 identifies a corresponding pedal position in the conversion table 211 as the current pedal position of the bicycle 910. Note that the tilt angles φ corresponding to the pedal positions at 3 and 9 o'clock are also detected on the 9 and 3 o'clock side. Therefore, a failure that the pedal positions at 3 and 9 o'clock are mistakenly identified may be avoided by always limiting the order of identifying the pedal positions to the order of 6, 3, 12, and 9 o'clock.

In the case of using a tilt sensor, a tilt sensor may be added to the sensor unit 930 in FIG. 7, and the above-described conversion may be done on the information terminal 920. In short, the position identifying unit 202 may be provided separately in the sensor unit 930 and the information terminal 920. Alternatively, a small-sized circuit that stores the conversion table 211 and performs the above-mentioned conversion may be provided in the sensor unit 930, and the position identifying unit 202 may be aggregated with the sensor unit 930.

The position identifying unit 202 transmits information representing the position of the muscle, which is identified using a rotation angle sensor motion or a tilt sensor, to the determination circuit 203 wirelessly, for example.

The determination circuit 203 is a circuit that refers to a determination reference that indicates the corresponding relationship between the position and shape of a muscle and the activity amount of the muscle, and determines the activity amount of the muscle using the position of the muscle identified by the position identifying unit 202 and the shape of the muscle obtained by the shape obtainer 201. The activity amount may be represented in terms of a numeral, or in terms of the degree or tendency merely indicating that the activity amount is great or small.

As the determination circuit 203, for example, a processor, a memory, and a communication circuit included in the information terminal 920 in FIG. 7 are used. The communication circuit receives information representing the shape and position of the muscle, transmitted from the shape obtainer 201 and the position identifying unit 202. The memory stores the above-mentioned determination reference. The muscle activity amount is determined by executing, by the processor, a program stored in the memory.

FIG. 9 is a diagram illustrating exemplary determination references 212 stored in the determination circuit 203. The determination references 212 in FIG. 9 indicate the contractile average and the relaxant average of the angle θa, obtained for the four pedal positions at 6, 3, 12, and 9 o'clock in the above-mentioned experiment. Here, the contractile average and the relaxant average corresponding to one pedal position are an exemplary determination reference that indicates the position (pedal position) of the muscle, the shape (angle θa) of the muscle, and the activity amount (contractile average or relaxant average) of the muscle in an associated manner. In short, the determination references 212 in FIG. 9 indicate four determination references corresponding to different positions of the muscle.

Note that the determination references 212 in FIG. 9 are suitable for determining the activity amount of the vastus lateralis muscle 111 where the shape is reflected in the angle θa (see FIGS. 1B and 1C), but application of such determination references is not limited to the vastus lateralis muscle 111. For example, determination references suitable for the angle θe or the angle θg may be set for determining the activity amount of the vastus medialis muscle 113 or the hamstring 114, and processing similar to that described below for the angle θa may be applied.

The determination circuit 203 receives the shape of the muscle (angle θa) from the shape obtainer 201, receives the position of the muscle (pedal position) from the position identifying unit 202, and selects a determination reference corresponding to the received position of the muscle (pedal position). The determination circuit 203 determines the muscle activity amount by referring to an angle range whose two ends are the contractile average and the relaxant average of the selected determination reference of the received shape of the muscle (angle θa).

Specifically, for example, in accordance with the following equation, the muscle activity amount may be determined in terms of percentage:

muscle activity amount=(angle θa−relaxant average)/(contractile average−relaxant average)×100 (1)

When the calculation result is less than 0, the result may be corrected to 0; and, when the calculation result is greater than 100, the result may be corrected to 100.

If it is only necessary to display the degree or tendency merely indicating that the activity amount is great or small, a threshold may be set (such as at a midpoint) between the contractile average and the relaxant average, and, if the difference between the angle θa and the contractile average is less than the difference between the threshold and the contractile average, it may be determined that the muscle activity amount is great. In other words, in the case of a determination reference where the contractile average is less than the relaxant average (when the pedal position is within a range including 6 or 3 o'clock), if the angle θa is less than the threshold, it is determined that the muscle activity amount is great. In the case of a determination reference where the contractile average is greater than the relaxant average (when the pedal position is within a range including 12 or 9 o'clock), if the angle θa is greater than the threshold, it is determined that the muscle activity amount is great.

The determination circuit 203 determines the muscle activity amount in accordance with the shape and position of the muscle, periodically received from the shape obtainer 201 and the position identifying unit 202, and notifies the outputter 204 of the result of determining the muscle activity amount.

The outputter 204 outputs the result of determining the muscle activity amount, sent from the determination circuit 203.

The outputter 204 may use, for example, a display included in the information terminal 920 in FIG. 7. The outputter 204 sends the muscle activity amount, sent from the determination circuit 203, as a visual feedback to the user 900 via the display.

Alternatively, the outputter 204 may use a loudspeaker. The outputter 204 sends the muscle activity amount as an aural feedback to the user 900 via the loudspeaker. Alternatively, the outputter 204 may output the activity amount to an information terminal and causes the information terminal to vibrate, thereby sending a tactile feedback to the user 900. For example, the information terminal vibrates when the muscle activity amount is greater than or equal to a certain amount.

Alternatively, the outputter 204 may output mechanically-readable data, including saving of the data in a memory or transmission of the data to an external device (not illustrated).

Next, the operation of the muscle activity amount determining device 200 will be described as an example of a muscle activity amount determining method according to the first embodiment.

FIG. 10 is a flowchart illustrating an example of the operation of the muscle activity amount determining device 200.

The shape obtainer 201 obtains the shape of a muscle of the user 900 (S11).

The position identifying unit 202 identifies the position of the muscle during certain exercise done by the user 900 (S12). For simplicity of explanation, it is assumed that obtaining of the shape is synchronous with identification of the position, and the shape and position of the muscle at substantially identical times are sent to the determination circuit 203.

The determination circuit 203 refers to a determination reference that indicates the corresponding relationship between the position and shape of the muscle and the activity amount of the muscle, and determines the activity amount of the muscle using the position of the muscle identified by the position identifying unit 202 and the shape of the muscle obtained by the shape obtainer 201 (S13).

The outputter 204 outputs the activity amount determined by the determination circuit 203 (S14).

By repeating steps S11 to S14 described above, the muscle activity amount at each of the pedal positions at 6, 3, 12, and 9 o'clock is determined every rotation in the pedaling.

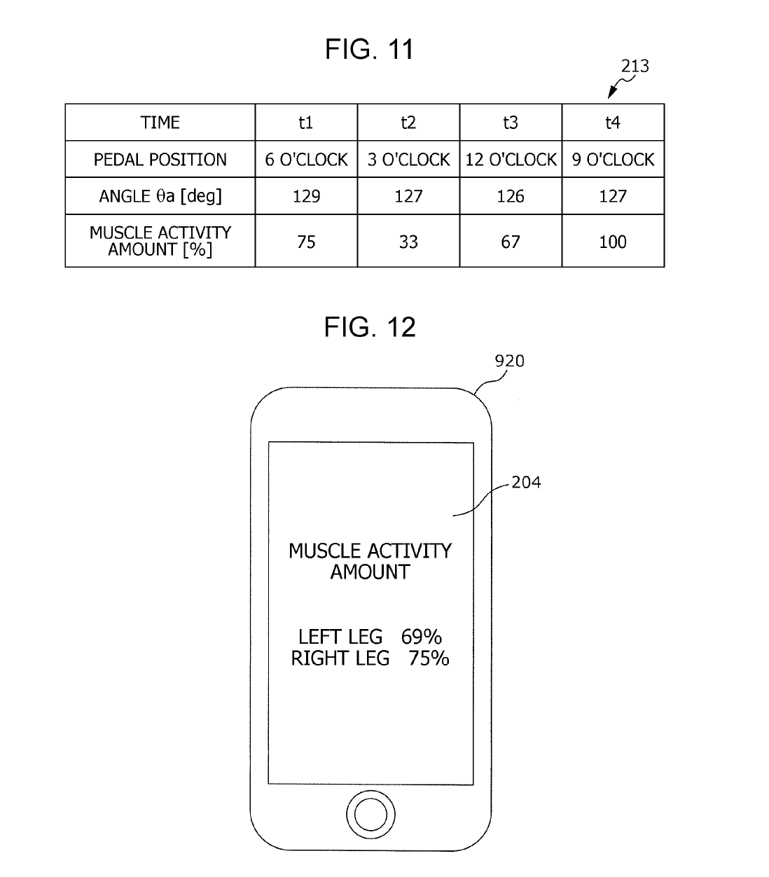

FIG. 11 is a diagram illustrating an example of the operation result of the muscle activity amount determining device 200, which indicates an execution result 213 of the flowchart in FIG. 10. In the execution result 213 in FIG. 11, the pedal position is identified as 6 o'clock at time t1, and 129 degrees is obtained as the angle θa. A determination reference corresponding to the pedal position at 6 o'clock in FIG. 9 is selected, reference is made to a contractile average of 126 degrees and a relaxant average of 138 degrees, and (129−138)/(126−138)×100 is calculated in accordance with equation (1) described above, thereby determining that the muscle activity amount is 75%. Likewise, the muscle activity amount is determined as 33%, 67%, and 100% at times t2, t3, and t4, respectively.

FIG. 12 is a diagram illustrating an exemplary output of the muscle activity amount. FIG. 12 illustrates an example in which the execution result 213 in FIG. 11 is displayed on the outputter 204, which is a liquid crystal display of the information terminal 920. As the muscle activity amount of the left leg in FIG. 12, 69%, which is the average of the muscle activity amounts at the pedal positions at 6, 3, 12, and 9 o'clock in FIG. 11, is displayed. The muscle activity amount of the right leg in FIG. 12 is also the average of the muscle activity amounts of the right leg in one rotation of pedaling, which is determined as in the case of the left leg.

### Advantageous Effects

According to the muscle activity amount determining device 200 and the muscle activity amount determining method described above, the muscle activity amount can be determined in accordance with the position and shape of a muscle. A sensor that detects the position and shape of a muscle is less likely to generate noise in a detection result even without certainly maintaining an electrical connection between the sensor and the skin of a user, unlike an electrode for measuring surface EMG. In other words, the muscle activity amount can be accurately determined using an easy-to-mount measuring instrument. By using the shape of a muscle, and the position of the muscle during exercise, the influence of deformation of the muscle due to gravity or an external force can be cancelled out, and the muscle activity amount can be more accurately determined.

As a result, a muscle activity amount determining device and a muscle activity amount determining method that use an easy-to-wear measuring instrument and that can highly accurately determine the activity amount of a muscle even during heavy exercise are provided.

### Second Embodiment

The configuration of the muscle activity amount determining device and the muscle activity amount determining method, which use no electrode of the related art at all for measuring surface EMG signals, is exemplary described in the first embodiment. However, it is not necessary to completely exclude the use of surface EMG, and the muscle activity amount may be determined using both the shape and position of a muscle and surface EMG.

In a second embodiment, a muscle activity amount determining device and a muscle activity amount determining method that use both the shape and position of a muscle and surface EMG will be described.

At first, an experiment for deriving the muscle activity amount determining device and the muscle activity amount determining method according to the second embodiment will be described.

In the first embodiment, whether the user is applying strength to the muscle of interest is determined using an angle formed by the portion of interest and the horizontal plane and the shape of the muscle. In the second embodiment, a method of more accurately measuring a muscle contraction force using surface EMG and the muscle shape will be described.

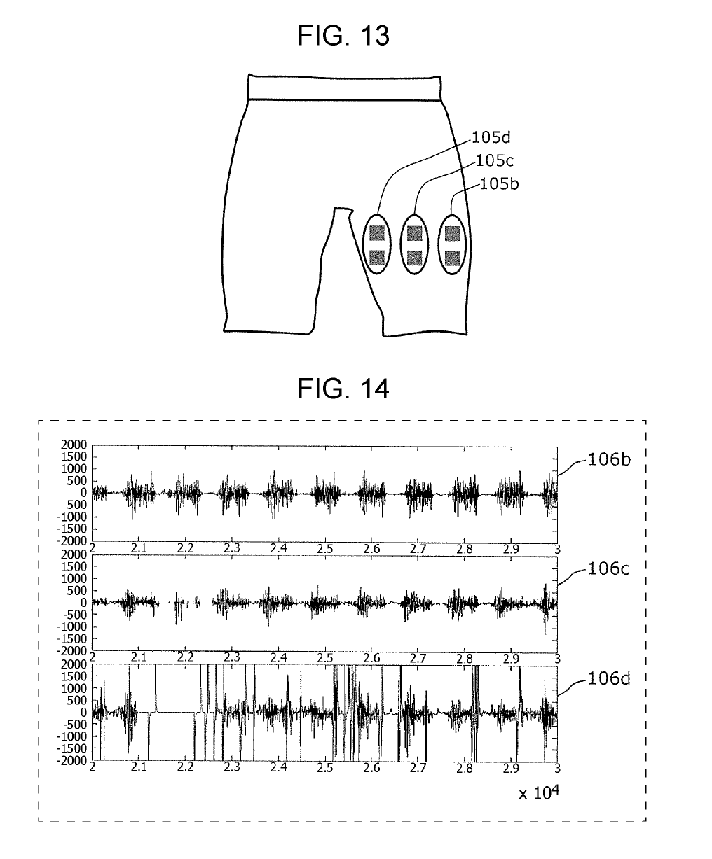

As illustrated in FIG. 13, the inventors of the present disclosure sew electrically conductive fabrics (hereinafter referred to as “fabric electrodes”) at six positions on a pair of shorts for bicycle, the six positions facing the quadriceps femoris muscle, thereby configuring electrode pairs 105b, 105c, and 105d. The size of each fabric electrode is 28 mm×28 mm, and an inter-electrode distance is 10 mm. Furthermore, in order to establish connect the electrodes of an EMG sensor, a flat surface of the male side of press-studs and the fabric electrodes are sewn together with thread, thereby connecting the electrodes of the EMG sensor for six channels from the outside when the user wears the shorts. Differential potentials between electrodes on an upper column and electrodes thereunder are measured as EMG signals.

The material of the fabric electrodes is polyester coated with copper and nickel, and the surface resistance thereof is 0.03 to 0.05Ω/□. As the EMG sensor, an EMG potential measuring device manufactured by Polymate is used. A high-pass filter that passes signals with a frequency higher than 10 Hz and a low-pass filter that passes signals with a frequency lower than 500 Hz are used, and the sampling frequency is 1000 Hz.

An experiment task will be described. A test subject is asked to pedal a bicycle out of the saddle for one minute after the test subject starts sweating. The rotation speed is 60 rpm (60 laps in one minute).

FIG. 14 is a graph illustrating exemplary EMG signals measured in the experiment. In FIG. 14, the abscissa axis represents time, and the ordinate axis represents the EMG potential. Here, a waveform 106b is an EMG signal measured around the vastus lateralis muscle by the electrode pair 105b. A waveform 106c is an EMG signal measured around the rectus femoris muscle by the electrode pair 105c. A waveform 106d is an EMG signal measured around the vastus medialis muscle by the electrode pair 105d. FIG. 14 illustrates that the waveform 106d is the most unstable, and the waveform 106b is the most stable.

One conceivable reason that an EMG signal becomes unstable is that the fabric electrodes move along with the stretch shorts, and accordingly the contact impedance changes. For example, because the electrode pair 105d is near the vastus medialis muscle, which is a place where the shorts are displaced most easily, the electrode pair 105d is unable to measure an EMG signal in a stable manner.

The experiment conducted by the inventors of the present disclosure demonstrates clearly that, when an EMG measuring wear incapable of fixing the electrodes to the skin is used, there are portions where electrode displacement is likely to occur and portions where electrode displacement is not likely to occur.

On the basis of the above-mentioned knowledge acquired from the experiment, the inventors of the present disclosure propose a muscle activity amount determining device and a muscle activity amount determining method using both a muscle shape sensor and an EMG sensor in portions where electrode displacement is likely to occur and portions where electrode displacement is not likely to occur.

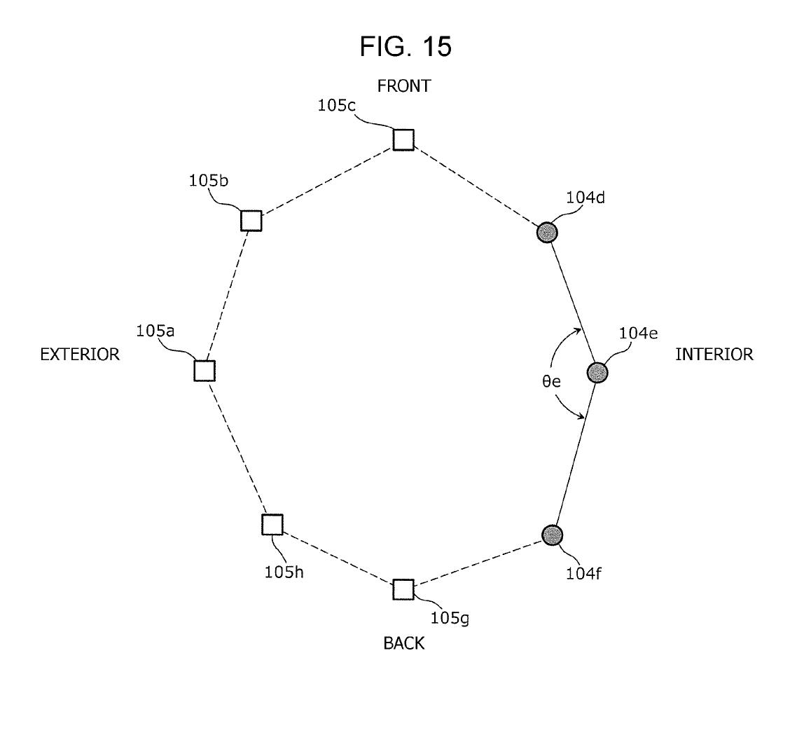

Specifically, as illustrated in FIG. 15, in the case of a thigh, because a portion around the vastus medialis muscle is a place where it is difficult to stably measure an EMG signal, it is effective to use a muscle shape sensor (such as the markers 104d to 104f) that is invulnerable to the influence of displacement. In a place where it is possible to stably measure an EMG signal (such as the vastus lateralis muscle), an EMG sensor (such as the electrode pairs 105a to 105c, 105g, and 105h) is used to measure surface EMG signals, and, using both the muscle shape and the surface EMG signals, the muscle activity amount can be more accurately determined.

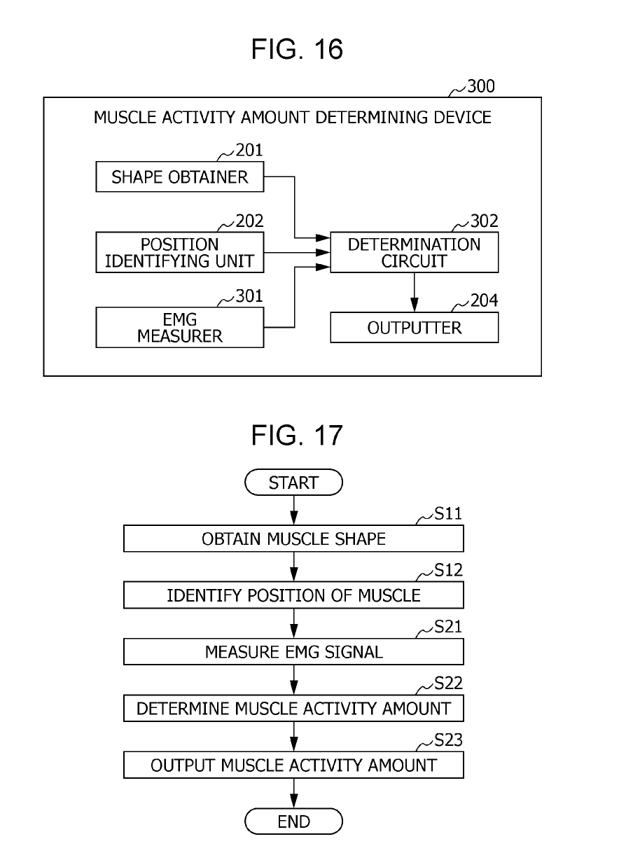

FIG. 16 is a block diagram illustrating an exemplary functional configuration of the muscle activity amount determining device according to the second embodiment. A muscle activity amount determining device 300 is a device that determines the activity amount of a muscle by using both the position and shape of the muscle during certain exercise, and surface EMG. The muscle activity amount determining device 300 is different from the muscle activity amount determining device 200 in FIG. 6 in the point that an EMG measurer 301 is added, and a determination circuit 302 is changed. Configurations different from the muscle activity amount determining device 200 will be mainly described below.

The EMG measurer 301 is a sensor that measures an EMG signal on a body surface near the muscle of interest of a user, and a general EMG sensor is used as the EMG measurer 301. The EMG measurer 301 outputs the measured EMG signal to the determination circuit 302.

The determination circuit 302 holds an EMG reference that indicates an EMG feature amount and a muscle activity (muscle contraction strength) in an associated manner. The EMG reference may be a reference on a user-by-user basis, or may be a normalized reference. The EMG feature amount may be, for example, the root-mean-square of an EMG signal. Specifically, the EMG feature amount may be the root-mean-square of an EMG signal obtained by preliminarily measuring a surface EMG signal of a user whose muscles are contracted with the maximal muscle strength.

The determination circuit 302 receives an EMG signal transmitted from the EMG measurer 301, and calculates an EMG feature amount (such as the root-mean-square) from the received EMG signal. Furthermore, the determination circuit 302 determines the ratio of the calculated root-mean-square to the root-mean-square of the maximal muscle strength indicated by the EMG reference as a muscle activity amount (muscle contraction strength) based on surface EMG.

In parallel with determination of the muscle activity amount based on surface EMG, the determination circuit 302 additionally determines a muscle activity amount based on the position and shape of the muscle, like the determination circuit 203.

The determination circuit 302 determines the activity amount of the overall muscle by using both the determined muscle activity amount based on surface EMG and the muscle activity amount based on the position and shape of the muscle. As an example of using both surface EMG and the position and shape of the muscle, the determination circuit 302 may determine the muscle activity amount of each muscle as, out of the muscle activity amount based on an EMG signal and the muscle activity amount based on the position and shape of the muscle, one of the muscle activity amounts in accordance with the type of sensor placed near the muscle. In addition, the determination circuit 302 may determine the average of the muscle activity amount based on an EMG signal and the muscle activity amount based on the position and shape of the muscle as the muscle activity amount of the overall muscle.

Next, the operation of the muscle activity amount determining device 300 will be described as an example of the muscle activity amount determining method according to the second embodiment. Steps different from the operation of the muscle activity amount determining device 200 will be mainly described below.

FIG. 17 is a flowchart illustrating an example of the operation of the muscle activity amount determining device 300.

The shape obtainer 201 obtains the shape of a muscle of a user (S11). The position identifying unit 202 identifies the position of the muscle during certain exercise done by the user (S12). Steps S11 and S12 are the same as those described above.

The EMG measurer 301 measures a surface EMG signal of the user while the user is doing exercise (EMG signal of the muscle of interest), and the measurement result is transmitted to the determination circuit 302 (S21).

The determination circuit 302 determines the muscle activity amount based on the position and shape of the muscle and the muscle activity amount based on the surface EMG signal, and determines the activity amount of the overall muscle by using both the muscle activity amounts (S22). The outputter 204 outputs the activity amount determined by the determination circuit 302 (S23).

### Advantageous Effects

According to the muscle activity amount determining device 300 and the muscle activity amount determining method described above, in addition to the advantageous effects described with regard to the muscle activity amount determining device 200, the muscle activity can be determined with yet a higher accuracy by additionally using surface EMG.

Although the muscle activity amount determining device and the muscle activity amount determining method according to one or more aspects of the present disclosure have been described on the basis of the embodiments, the present disclosure is not limited to these embodiments. The one or more aspects of the present disclosure may include an embodiment obtained by adding various modifications conceivable by those skilled in the art to the embodiments or an embodiment constructed by combining elements in different embodiments without departing from the scope of the present disclosure.

The muscle activity amount determining device and the muscle activity amount determining method according to the present disclosure are usable in various scenes where there is a need to determine the muscle activity amount while a user is exercising, such as in training dedicated for a particular competition, general exercise, or rehabilitation.

What is claimed is:

1. A muscle activity amount determining device comprising:
a shape obtainer that obtains a shape of a muscle of a user when the user is doing certain exercise;
a position identifying unit that identifies a position of the muscle of the user when the user is doing the exercise;
a determination circuit that refers to a determination reference that indicates a corresponding relationship between the position and shape of the muscle and an activity amount of the muscle, and determines the activity amount of the muscle using the position of the muscle identified by the position identifying unit and the shape of the muscle obtained by the shape obtainer; and
an outputter that outputs the activity amount of the muscle, the activity amount being determined by the determination circuit.

2. The muscle activity amount determining device according to claim 1, wherein the certain exercise is pedaling, which is the user's turning pedals.

3. The muscle activity amount determining device according to claim 1, wherein:
the muscle is included in a leg of the user,
the shape obtainer obtains the shape of the muscle in a cross-section including the muscle of the leg of the user, and
the determination circuit determines the activity amount of the muscle using the shape of the muscle.

4. The muscle activity amount determining device according to claim 1, wherein:
the shape obtainer obtains, as the shape of the muscle, an angle formed by a polygonal line connecting three points in a cross-section of a leg of the user using sensors placed on the leg, and
the determination circuit determines the activity amount of the muscle using the angle.

5. The muscle activity amount determining device according to claim 1, wherein, using a first sensor, a second sensor, and a third sensor placed on a leg of the user, the shape obtainer obtains, as the shape of the muscle, an angle formed by a first straight line connecting the first sensor and the second sensor and a second straight line connecting the first sensor and the third sensor.

6. The muscle activity amount determining device according to claim 1, wherein the position identifying unit identifies the position of the muscle based on a tilt angle of a thigh including the muscle of the user from a horizontal plane.

7. The muscle activity amount determining device according to claim 2, wherein the position identifying unit obtains a rotation angle from a rotation angle sensor placed on one of the pedals, and identifies the obtained angle of the pedal as the position of the muscle.

8. The muscle activity amount determining device according to claim 1, wherein the determination circuit selects one determination reference from a plurality of determination references in accordance with the position of the muscle, refers to the selected determination reference, and determines the activity amount of the muscle using the shape of the muscle.

9. The muscle activity amount determining device according to claim 8, wherein the determination circuit selects different determination references from the plurality of determination references depending on whether the position of the muscle is within a certain range.

10. The muscle activity amount determining device according to claim 4, wherein:
when the position of the muscle during the exercise is within a first range, the determination circuit determines that the activity amount is great if the angle is greater than a threshold, and
when the position of the muscle during the exercise is within a second range different from the first range, the determination circuit determines that the activity amount is great if the angle is less than a threshold.

11. The muscle activity amount determining device according to claim 4, wherein:
when the position of the muscle during the exercise is within a third range, the determination circuit determines that the activity amount is great if the angle is less than a threshold, and
when the position of the muscle during the exercise is within a fourth range different from the third range, the determination circuit determines that the activity amount is great if the angle is less than a threshold.

12. A muscle activity amount determining method comprising:
obtaining, with the use of a shape obtainer, a shape of a muscle of a user when the user is doing exercise;
identifying, with the use of a position identifying unit, a position of the muscle of the user when the user is doing the exercise;
referring to, with the use of a determination circuit, a determination reference that indicates a corresponding relationship between the position and shape of the muscle and an activity amount of the muscle, and determining the activity amount of the muscle using the position of the muscle identified by the position identifying unit and the shape of the muscle obtained by the shape obtainer; and
outputting, with the use of an outputter, the activity amount of the muscle, the activity amount being determined by the determination circuit.

13. A non-transitory computer-readable recording medium storing a control program for causing a device with a processor to execute a process, the process comprising:
obtaining, with the use of a shape obtainer, a shape of a muscle of a user when the user is doing certain exercise;
identifying, with the use of a position identifying unit, a position of the muscle of the user when the user is doing the exercise;
referring to, with the use of a determination circuit, a determination reference that indicates a corresponding relationship between the position and shape of the muscle and an activity amount of the muscle, and determining the activity amount of the muscle using the position of the muscle identified by the position identifying unit and the shape of the muscle obtained by the shape obtainer; and
outputting, with the use of an outputter, the activity amount of the muscle, the activity amount being determined by the determination circuit.

14. A method comprising:
receiving location data indicating three different locations on a skin of a body region and data indicating an angle between the body region and a predetermined line, muscles being under the skin;
determining muscle activity information of the muscles based on the location data and the data; and
outputting the muscle activity information,
wherein a muscle angle is provided between a first line and a second line, the first line being provided on a first location and a second location, and the second line being provided on the second location and a third location,
wherein the first location, the second location, and the third location are the three different locations, a length on the skin between the first location and the second location is shorter than a length on the skin between the first location and the third location, and a length on the skin between the second location and the third location is shorter than the length on the skin between the first location and the third location,
wherein the muscle angle is a first muscle angle if the location data is first location data indicating first three different locations, the muscle angle is a second muscle angle if the location data is second location data indicating second three different locations, the muscle angle is a third muscle angle if the location data is third location data indicating third three different locations, and the muscle angle is a fourth muscle angle if the location data is fourth location data indicating fourth three different locations,
wherein the muscle activity information is first muscle activity information if the location data is the first location data and the data is first angle data indicating a first angle, the muscle activity information is second muscle activity information if the location data is the second location data and the data is the first angle data, the muscle activity information is third muscle activity information if the location data is the third location data and the data is second angle data indicating a second angle different from the first angle, and the muscle activity information is fourth muscle activity information if the location data is the fourth location data and the data is the second angle data,
wherein the first muscle angle is larger than the second muscle angle, and the first muscle activity information indicates that the muscles are more relaxed than the second muscle activity information, and
wherein the third muscle angle is larger than the fourth muscle angle, and the fourth muscle activity information indicates that the muscles are more relaxed than the third muscle activity information.

15. The method according to claim 14,
wherein the first angle is a maximum angle between the body region and the predetermined line and the second angle is a minimum angle between the body region and the predetermined line,
wherein the predetermined line is a horizontal line,
wherein the body region is a thigh,
wherein a camera obtains images, each of the images including images of three marks put on the skin, thereby the location data being provided, and
wherein the first location data, the second location data, the third location data, and the fourth location data correspond to the images, respectively.
